(19) United States
(12) Patent Application Publication
(10) Pub. No.: US 2009/0092661 A1
(43) Pub. Date: Apr. 9, 2009

(54) LIPOSOME COMPOSITIONS FOR IN VIVO ADMINISTRATION OF BORONIC ACID COMPOUNDS

(76) Inventors: Anthony Huang, Saraloga, CA (US); Bing Luo, Fremont, CA (US); Jinkang Wang, San Francisco, CA (US); Yuanpeng Zhang, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/195,755

(22) Filed: Aug. 21, 2008

Related U.S. Application Data
(60) Provisional application No. 60/957,045, filed on Aug. 21, 2007.

(51) Int. Cl. A61K 9/127

(52) U.S. Cl. 424/450

(57) ABSTRACT

Liposome formulations for administration of a boronic acid compound are described. The liposomes are comprised of a phospholipid having two acyl chains with between 20-22 carbon atoms in each chain and a boronic acid compound entrapped in the liposomes. In a preferred embodiment, the boronic acid compound is in the form of a complex with meglumine.

### CROSS REFERENCE TO RELATED APPLICATIONS

The present application claims priority to U.S. Provisional Patent Application Ser. No. 60/957,045, filed on Aug. 21, 2007, which is hereby incorporated by reference.

### TECHNICAL FIELD

The subject matter described herein relates to a liposome formulation having an entrapped boronic acid compound. More particularly, the subject matter relates to liposomes prepared from components that improve loading and retention of a peptide boronic acid compound within the liposomes.

### BACKGROUND

Liposomes, or lipid bilayer vesicles, are spherical vesicles comprised of concentrically ordered lipid bilayers that encapsulate an aqueous phase. Liposomes serve as a delivery vehicle for therapeutic and diagnostic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, i.e., so-called STEALTH® or long-circulating liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Ideally, such liposomes can be prepared to include an entrapped therapeutic or diagnostic compound (i) with high loading efficiency, (ii) at a high concentration of entrapped compound, and (iii) in a stable form, i.e., with little compound leakage during storage.

### BRIEF SUMMARY

The following aspects and embodiments thereof described and illustrated below are meant to be exemplary and illustrative, not limiting in scope. In one aspect, a liposome formulation comprising liposomes comprised of a phospholipid having two acyl chains with between 20-22 carbon atoms in each chain and a boronic acid compound entrapped in the liposomes is provided. The boronic acid compound is in the form of a complex with meglumine.

In one embodiment, the phospholipid is an asymmetric phospholipid.

In another embodiment, the phospholipid is a symmetric phospholipid.

In one embodiment, the phospholipid has 20 carbon atoms. In yet another embodiment, the phospholipid is a saturated phospholipid.

In still another embodiment, the phospholipid is selected from the group consisting of phosphatidylcholine, phosphatidyethanolamine, phosphatidic acid, and phosphatidylinositol.

In a preferred embodiment, the phospholipid is 1,2-arachidoyl-sn-glycero-3-phosphocholine (DAPC) or 1,2-dibehenoyl-sn-glycero-3-phosphocholine (DBPC).

In another embodiment, the liposomes further include a phospholipid covalently attached to a hydrophilic polymer. An exemplary hydrophilic polymer is polyethylene glycol.

In yet another embodiment, the phospholipid covalently attached to a hydrophilic polymer is distearoylphosphatidylethanolamine-polyethylene glycol.

In one embodiment, the boronic acid compound is a peptide boronic acid compound. In yet another embodiment, the boronic acid compound is bortezomib.

The formulation, in another embodiment, comprises liposomes that further comprise entrapped acetic acid.

In another aspect, a method for preparing liposomes having an entrapped boronic acid compound is provided. The method comprises providing liposomes comprised of a phospholipid having two acyl chains, each having between 20-22 carbon atoms, the liposomes having meglumine entrapped therein; and incubating the liposomes in the presence of a boronic acid compound at a temperature lower than the phase transition temperature of the phospholipid. The incubating is effective to achieve uptake of the boronic acid compound into the liposomes.

In one embodiment, the liposomes are comprised of a phospholipid selected from the group consisting of phosphatidylcholine, phosphatidyethanolamine, phosphatidic acid, and phosphatidylinositol.

In another embodiment, incubating is effective to achieve uptake of greater than 90% of the boronic acid compound into the liposomes.

In still another aspect, an improvement in a method of preparing a liposome composition comprised of liposomes comprised of a phospholipid having two acyl chains with between 20-22 carbon atoms in each chain and a boronic acid compound entrapped in the liposomes is provided. The improvement comprises loading the boronic acid compound into the liposomes by incubating liposomes and the boronic acid compound at a temperature below the phase transition temperature.

In one embodiment, the improvement further comprises forming, prior to said incubating, liposomes that comprise meglumine entrapped therein.

In another embodiment of the improvement, the phospholipid is 1,2-arachidoyl-sn-glycero-3-phosphocholine (DAPC) and the loading is at a temperature of between about 25-50° C.

In still another embodiment, the phospholipid is 1,2-dibehenoyl-sn-glycero-3-phosphocholine (DBPC) and said loading is at a temperature of between about 25-50° C.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1H show the structures of exemplary peptide boronic acid compounds;

FIG. 2 illustrates loading of an exemplary peptide boronic acid into a liposome against a higher inside/lower outside pH gradient for formation inside the liposome of a boronate ester compound with a polyol;

FIGS. 3A-3C shows the structures of the polyols sorbitol (FIG. 3A), alfa-glycoheptonic acid (also referred to as glucoheptonate or gluceptate; FIG. 3B), and meglumine (FIG. 3C);

FIG. 4A shows the absorbance at 270 nm for column fractions for liposomes (HSPC/CHOL/mPEG-DSPE 50:45:5 mol/mol) containing entrapped meglumine incubated in the presence of bortezomib at 65° C. for 30 minutes (diamonds), 60 minutes (squares), or 120 minutes (triangles), the peak at fraction number 10 corresponding to unentrapped drug;

FIG. 4B shows the absorbance at 270 nm for column fractions for liposomes (HSPC/CHOL/mPEG-DSPE 50:45:5 mol/mol) containing entrapped meglumine incubated in the presence of bortezomib at 20-25° C., the peak at fraction number 4 corresponding to liposome entrapped drug;

FIG. 5 shows the absorbance at 270 nm for gel-filtration column fractions for liposomes containing entrapped meglumine and acetic acid incubated in the presence of bortezomib at 20-25° C., the peak between fraction numbers 14-18 corresponding to liposome entrapped drug and fractions 35-50 corresponding to unentrapped drug fractions;

FIG. 6 shows the concentration, in ng/mL, of bortezomib in the plasma of mice as a function of time, in hours, following administration of bortezomib entrapped in liposomes comprised of HSPC/cholesterol/mPEG-DSPE (50:45:5 mol/mol), with meglumine/acetic acid as the complexing agent, where bortezomib was administered at doses of 0.53 mg/mL (triangles), 1.04 mg/mL (squares) and 2.13 mg/mL (triangles);

FIG. 7 shows the concentration, in μg/mL, of bortezomib in whole blood in vitro as a function of incubation time, in hours, for liposomes comprised of the lipids egg sphingomyelin/cholesterol (circles), egg sph ingomyel in/cholesterol/m PEG-DSPE (triangles), or egg sphingomyelin (diamonds);

FIGS. 8A-8B show the concentration, in μg/mL, of bortezomib in whole blood in vitro as a function of incubation time, in hours, at 17° C. (FIG. 8A) or at 37° C. (FIG. 8B) for liposomes comprised of HSPC/mPEG-DSPE (95/5, triangles) or 1,2-diarachidoyl-sn-glycero-3-phosphocholine (C20:0 PC)/mPEG-DSPE (95/5, diamonds);

FIG. 9 shows the concentration, in μg/mL, of bortezomib in plasma as a function of time, in hours, following intravenous administration to mice of liposomes comprised of C20:0PC/mPEG-DSPE (95/5, squares), 1,2-dibehenoyl-sn-glycero-3-phosphocholine (C22:0PC/mPEG-DSPE (95/5, triangles), 1,2-dilignoceroyl-sn-glycero-3-phosphocholine (C24:0PC/mPEG-DSPE (95/5, triangles and squares)) or following administration of free drug (diamonds);

FIG. 10A shows the percent bortezomib encapsulation in liposomes composed of C22:0PC/mPEG-DSPE (95/5) as a function of time, in weeks, when stored at 5° C. (diamonds) or at 25° C. (squares);

FIG. 10B shows the percent bortezomib encapsulation in liposomes composed of C22:0PC/mPEG-DSPE (95/5, diamonds, squares) or of C24:0PC/mPEG-DSPE (95/5, triangles, circles) as a function of time, in weeks, when stored at 4° C. (diamonds, triangles) or at 25° C. (squares, circles);

FIGS. 11A-11C show the concentration of bortezomib, in ng/mL, as a function of time, in hours, after administration to mice intravenously, the drug concentration in plasma (FIG. 11A), blood (FIG. 11B) and tumor (FIG. 11C) for the drug in free form (diamonds) or entrapped in liposomes (C22:0 PC/mPEG 95:5) (squares);s

FIG. 12 shows the plasma concentration of bortezomib, in ng/mL, as a function of time, in hours, after administration to mice intravenously in liposome-entrapped form (C22:0 PC/mPEG 95:5) (solid circles) or in free form (open circles); and

FIG. 13 shows the percent bortezomib remaining in plasma as a function of time, in hours, following administration of Formulations 4 and 5 (Example 6) in normal rats.

FIG. 14 shows the tumor size of mice bearing xenograft CWR22 tumors, as a function of time, in days, in mice treated with free drug (triangles), liposome vehicle placebo (squares), bortezomib liposome formulations nos. 4 and 5 (inverted triangles, circles, respectively Example 6), and another liposome formulation (diamonds), administered weekly for four weeks at the time points indicated by arrows along the time axis.

### DETAILED DESCRIPTION

### I. Definitions

“Polyol” intends a compound having more than one hydroxyl (—OH) group per molecule. The term includes monomeric and polymeric compounds containing alcoholic hydroxyl groups such as sugars, glycerol, polyethers, glycols, polyesters, polyalcohols, carbohydrates, catecols, copolymers of vinyl alcohol and vinyl amine, etc.

“Peptide boronic acid compound” intends a compound of the form

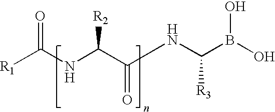

where R¹, R², and R³ are independently selected moieties that can be the same or different from each other, and n is from 1-8, preferably 1-4.

A “hydrophilic polymer” intends a polymer having some amount of solubility in water at room temperature. Exemplary hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers. A preferred hydrophilic polymer chain is polyethyleneglycol (PEG), preferably as a PEG chain having a molecular weight between 500-10,000 daltons, more preferably between 750-10,000 daltons, still more preferably between 750-5,000 daltons.

“Higher inside/lower outside pH gradient” refers to a transmembrane pH gradient between the interior of liposomes (higher pH) and the external medium (lower pH) in which the liposomes are suspended. Typically, the interior liposome pH is at least 1 pH unit greater than the external medium pH, and preferably 2-4 units greater.

“Liposome entrapped” intends refers to a compound being sequestered in the central aqueous compartment of liposomes, in the aqueous space between liposome lipid bilayers, or within the bilayer itself.

### II. Liposome Formulation

In one aspect, a liposome composition having an entrapped peptide boronic acid compound is provided. The liposomes include components that enhance loading and retention of the compound in the liposomes. The liposome composition and method of preparation are described in this section.

A. Liposome Components

The liposome formulation is comprised of liposomes containing an entrapped peptide boronic acid compound. Peptide boronic acid compounds are peptides containing an α-aminoboronic acid at the acidic, or C-terminal, end of the peptide sequence. In general, peptide boronic acid compounds are of the form:

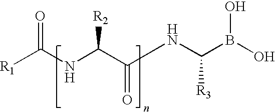

where R¹, R², and R³ are independently selected moieties that can be the same or different from each other, and n is from 1-8, preferably 1-4. Compounds having an aspartic acid or glutamic acid residue with a boronic acid as a side chain are also contemplated.

Preferably, R¹, R², and R³ are independently selected from hydrogen, alkyl, alkoxy, aryl, aryloxy, aralkyl, aralkoxy, cycloalkyl, or heterocycle; or any of R¹, R², and R³ may form a heterocyclic ring with an adjacent nitrogen atom in the peptide backbone. Alkyl, including the alkyl component of alkoxy, aralkyl and aralkoxy, is preferably 1 to 10 carbon atoms, more preferably 1 to 6 carbon atoms, and may be linear or branched. Aryl, including the aryl component of aryloxy, aralkyl, and aralkoxy, is preferably mononuclear or binuclear (i.e. two fused rings), more preferably mononuclear, such as benzyl, benzyloxy, or phenyl. Aryl also includes heteroaryl, i.e. an aromatic ring having one or more nitrogen, oxygen, or sulfur atoms in the ring, such as furyl, pyrrole, pyridine, pyrazine, or indole. Cycloalkyl is preferably 3 to 6 carbon atoms. Heterocycle refers to a non-aromatic ring having one or more nitrogen, oxygen, or sulfur atoms in the ring, preferably a 5- to 7-membered ring having include 3 to 6 carbon atoms. Such heterocycles include, for example, pyrrolidine, piperidine, piperazine, and morpholine. Either of cycloalkyl or heterocycle may be combined with alkyl; e.g. cyclohexylmethyl.

Any of the above groups (excluding hydrogen) may be substituted with one or more substituents selected from halogen, preferably fluoro or chloro; hydroxy; lower alkyl; lower alkoxy, such as methoxy or ethoxy; keto; aldehyde; carboxylic acid, ester, amide, carbonate, or carbamate; sulfonic acid or ester; cyano; primary, secondary, or tertiary amino; nitro; amidino; and thio or alkylthio. Preferably, the group includes at most two such substituents.

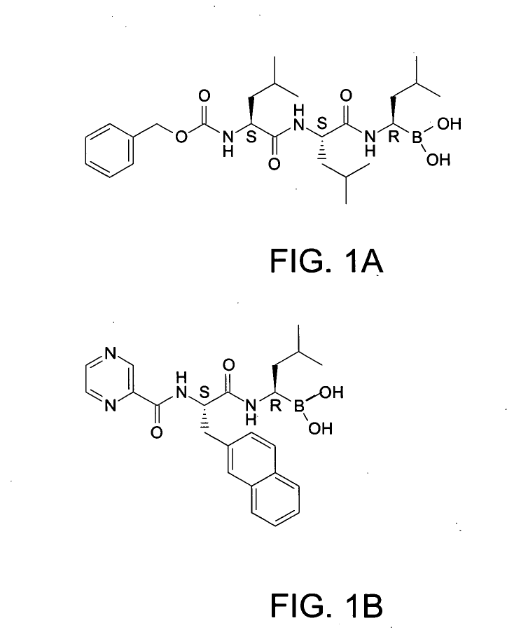

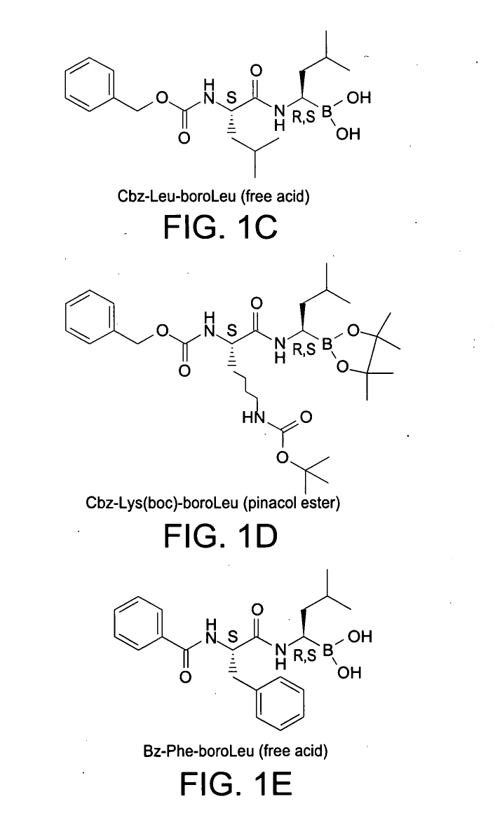

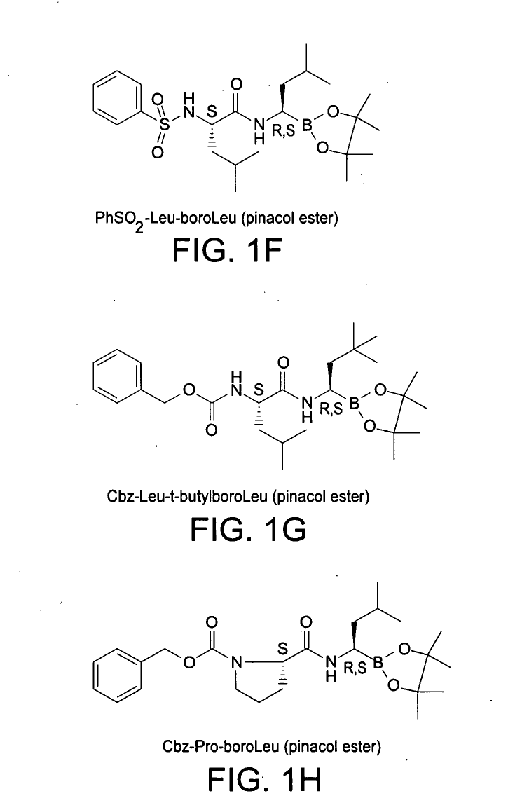

Exemplary peptide boronic acid compounds are shown in FIGS. 1A-1H. Specific examples of R¹, R², and R³ shown in FIGS. 1A-H include n-butyl, isobutyl, and neopentyl(alkyl); phenyl or pyrazyl(aryl); 4-((t-butoxycarbonyl)amino)butyl, 3-(nitroamidino)propyl, and (1-cyclopentyl-9-cyano)nonyl (substituted alkyl); naphthylmethyl and benzyl(aralkyl); benzyloxy(aralkoxy); and pyrrolidine (R² forms a heterocyclic ring with an adjacent nitrogen atom).

In general, the peptide boronic acid compound can be a mono-peptide, di-peptide, tri-peptide, or a higher order peptide compound. Other exemplary peptide boronic acid compounds are described in U.S. Pat. Nos. 6,083,903, 6,297,217, and 6,617,317, which are incorporated by reference herein.

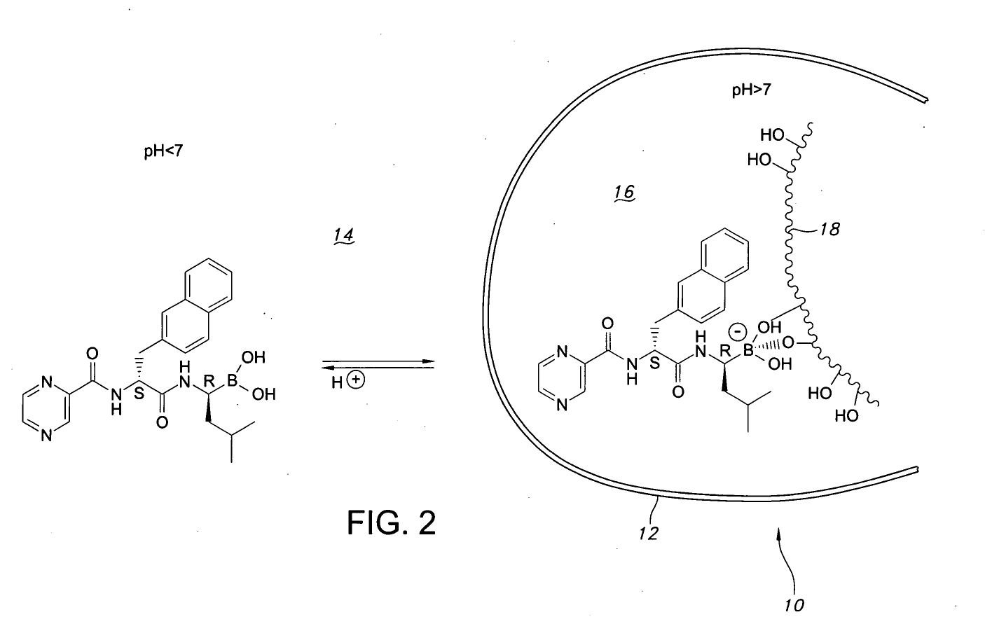

The peptide boronic acid compound is loaded into liposomes, to yield a liposome formulation where the peptide boronic acid compound is entrapped in the liposome in the form of a peptide boronate ester, according to the procedure illustrated in FIG. 2. FIG. 2 shows a liposome 10 having a lipid bilayer membrane represented by a single solid line 12. It will be appreciated that in multilamellar liposomes the lipid bilayer membrane is comprised of multiple lipid bilayers with intervening aqueous spaces. Liposome 10 is suspended in an external medium 14, where the pH of the external medium is about 7.0, generally between about 5.5-8.0, more generally between 6.0-7.0. Liposome 10 has an internal aqueous compartment 16 defined by the lipid bilayer membrane. Entrapped within the internal aqueous compartment is a polyol 18. The polyol is preferably a moiety having a cis 1,2- or a 1,3-diol functionality, and in a preferred embodiment the polyol is meglumine. The pH of the internal aqueous compartment is preferably greater than about 8.0, more preferably greater than 9, still more preferably greater than 10.

Also entrapped in the liposome is a peptide boronic acid compound, represented in FIG. 2 by bortezomib. Bortezomib is also shown in the external aqueous medium, prior to passage across the lipid bilayer membrane. In the external aqueous medium, the compound is mostly uncharged, due to the pH is significantly lower than the pKa=9.7 (calculated by ACD/labs version 6.0) for the boronic group. In its uncharged state, the compound is freely permeable across the lipid bilayer, because the compounds are rather lipophilic (log P=2.45+1.06, calculated by ACD/labs version 6.0). Formation of a boronate ester shifts the equilibrium to cause additional compound to permeate from the external medium across the lipid bilayer, leading to accumulation of the compound in the liposome. In another embodiment, the lower pH in the external suspension medium and the somewhat higher pH on the liposomal interior, combined with the polyol inside the liposome, induces drug accumulation into the liposome's aqueous internal compartment. Once inside the liposome, the compound reacts with the polyol to form a boronate ester. The boronate ester is essentially unable to cross the liposome bilayer, so that the drug compound, in the form of a boronate ester, accumulates inside the liposome. The stability of the boronic ester complex increases with increasing pH.

The concentration of polyol inside the liposomes is preferably such that the concentration of charged groups, e.g., hydroxyl groups, is significantly greater than the concentration of boronic acid compound. In a composition having a final drug concentration of 25 mM (internal drug concentration at 0.2 mg/mL total drug concentration), for example, the internal compound concentration of the polymer charge groups will typically be at least this great, preferably several fold of the drug concentration.

The polyol is present at a high-internal/low-external concentration; that is, there is a concentration gradient of polyol across the liposome lipid bilayer membrane. If the polyol trapping agent is present in significant amounts in the external bulk phase, the polyol reacts with the peptide boronic acid compound in the external medium, slowing accumulation of the compound inside the liposome. Thus, preferably, the liposomes are prepared, as described below, so that the composition is substantially free of polyol trapping agent in the bulk phase (outside aqueous phase).

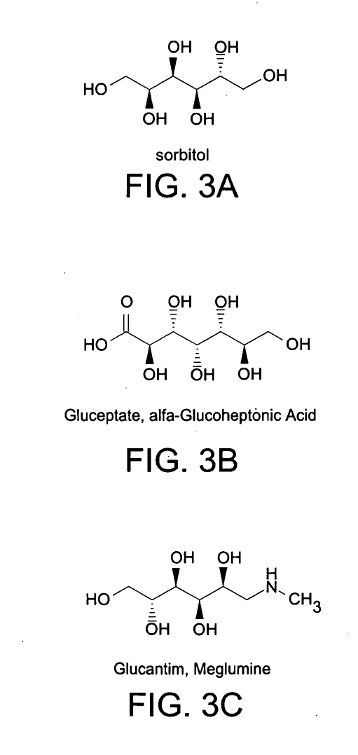

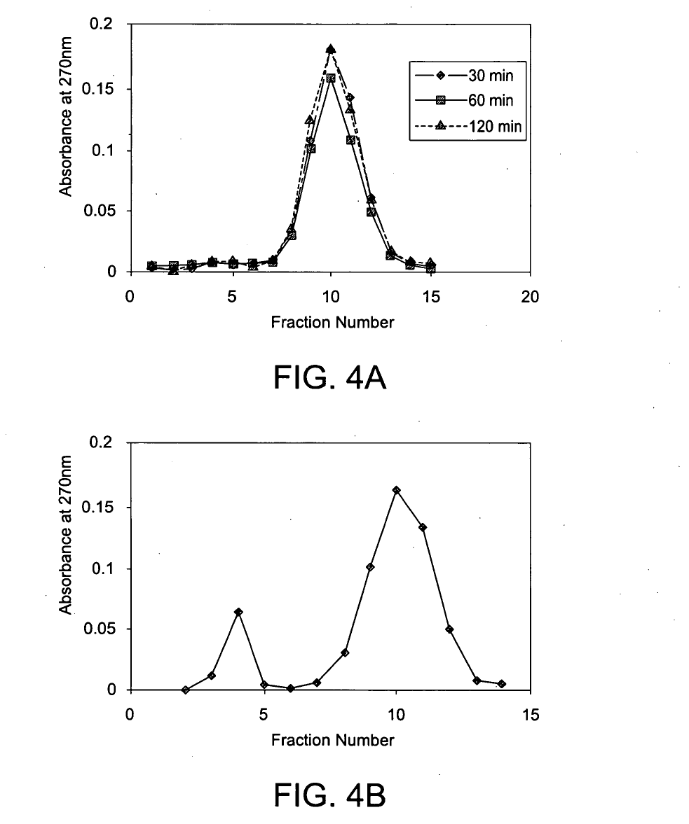

In supporting studies described herein, the exemplary compound bortezomib was loaded into liposomes having as a trapping agent (also referred to as a complexing agent) sorbitol, gluceptate, or meglumine. The structures of these compounds are shown in FIGS. 3A-3C, respectively. As set forth in Examples 1-3, liposomes were prepared using one of these complexing agents in the hydration buffer. After removal of any unentrapped complexing agent by dialysis, bortezomib was loaded into the liposomes by incubating the liposomes with a solution of drug at various temperatures for various times. No detectable drug was loaded into liposomes when sorbitol or gluceptate were present in the liposomes as the complexing reagent and loading was conducted at 60-65° C. A similar result was observed when meglumine was used as the complexing agent and loading was conducted at 65° C. This is illustrated by the data presented in FIG. 4A, which shows the absorbance at 270 nm for G10 desalting column fractions for liposomes containing entrapped meglumine incubated in the presence of bortezomib at 65° C. for 30 minutes (circles), 60 minutes (squares), or 120 minutes (triangles). The peak at fraction number 10 corresponds to unentrapped drug. However, and as seen in FIG. 4B, when the incubation was conducted at room temperature of about 20-25° C., bortezomib was loaded and retained in the liposomes, as evidenced by the peak at fraction number 4.

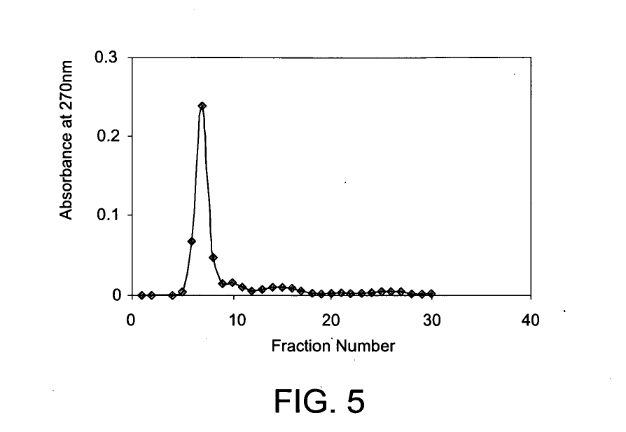

In another study, described in Example 4, bortezomib was loaded into liposomes against an ion gradient established by the presence of meglumine and acetic acid inside the liposomes. Addition of acetic acid to the internal hydration medium results in a high encapsulation efficiency of bortezomib, as seen in FIG. 5. In FIG. 5 the peak between fraction numbers 14-18 corresponds to liposome entrapped drug, and shows that about 95% of the total drug was entrapped in the liposomes by remote loading.

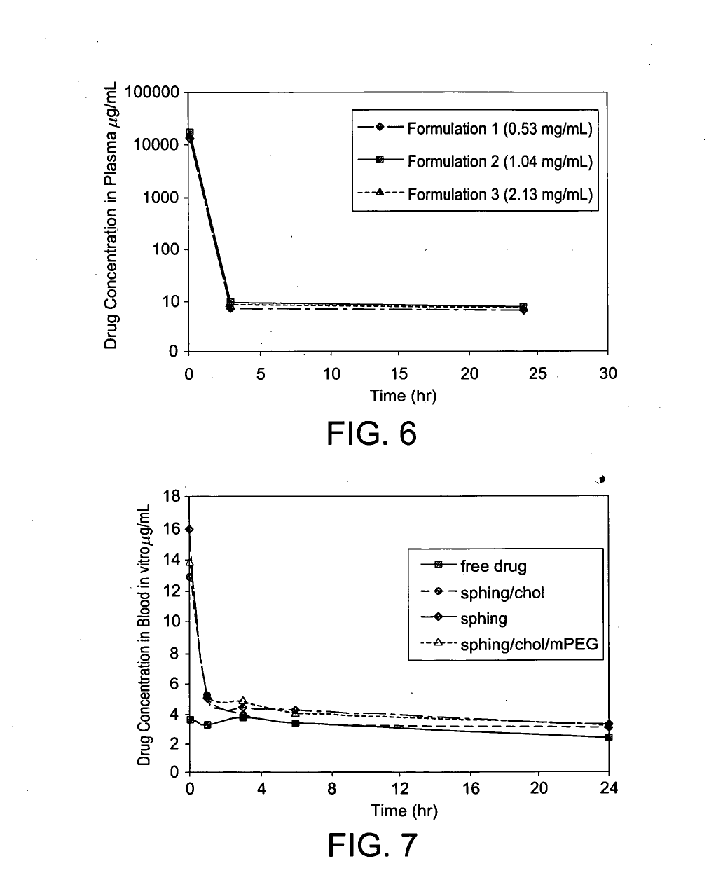

Liposomes having bortezomib entrapped by loading against a meglumine/acetic acid gradient were prepared to have drug concentrations of 0.5 mg/mL, 1.0 mg/mL, and 2.1 mg/mL, as described in Example 5. The three formulations were injected into mice at a drug dose of 1.6 mg/kg and the blood plasma concentration of bortezomib was determined as a function of time. FIG. 6 shows the concentration, in ng/mL, of bortezomib in the blood plasma of mice as a function of time, in hours, following administration of bortezomib entrapped in liposomes at drug concentrations of 0.53 mg/mL (triangles), 1.04 mg/mL (squares) and 2.13 mg/mL (triangles). Upon in vivo administration, the drug rapidly leaked from the liposomes, and at the three hour time point the plasma drug concentration was about the same as expected for in vivo administration of free bortezomib.

Further studies were performed to arrive at a liposome composition with improved in vivo retention of the boronic acid compound. As described in Example 6, liposomes were prepared from different lipid compositions and tested in an in vitro release assay using rat whole blood. Liposomes having a lipid bilayer comprised of egg sphingomyelin/cholesterol (95/5), egg sphingomyelin/cholesterol/mPEG-DSPE (50/45/5), or egg sphingomyelin were prepared and loaded with bortozemib (Example 6A). Release of the drug from the liposomes was analyzed using an in vitro release assay using whole rat blood. As seen in FIG. 7, the drug was rapidly released from liposomes comprised of egg sph ingomyel in/cholesterol (circles), egg sph ingomyel in/cholesterol/m PEG-DSPE (triangles), and egg sphingomyelin (diamonds).

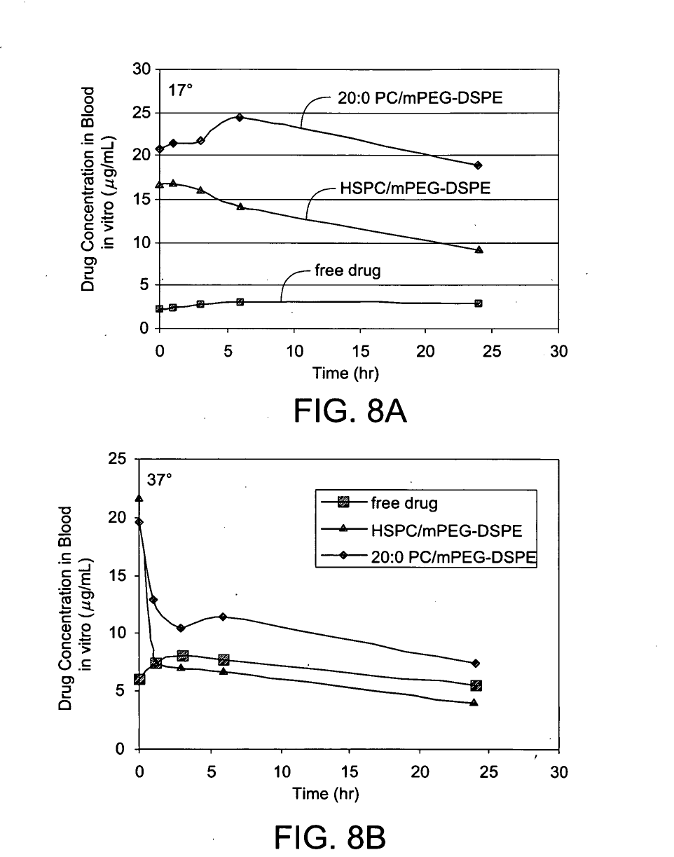

Liposomes having a lipid bilayer comprised of the phospholipid phosphocholine were prepared, the phosphocholine having acyl-chain lengths of 18, 20, 22, or 24 carbon atoms (Example 6B). FIGS. 8A-8B show the release of bortezomib from liposomes comprised of hydrogenated soy phosphocholine (C18:0; HSPC)/cholesterol/mPEG-DSPE (50:45:5, triangles) or of 1,2-diarachidoyl-sn-glycero-3-phosphocholine (20:0PC)/mPEG-DSPE (95/5, diamonds) at 17° C. (FIG. 8A) or at 37° C. (FIG. 8B). The data in FIGS. 8A-8B shows that liposomes prepared with the C20:0PC lipid retained the drug noticeably better when incubated in blood for a longer period of time, relative to liposomes prepared with the C18:0PC lipid.

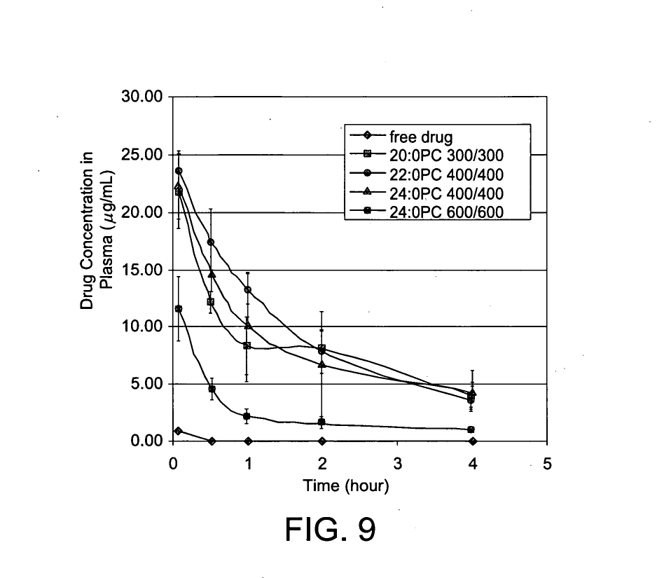

The liposomes prepared according to Example 6B were administered via intravenous injection to mice. Blood samples were taken over a four hours period post injection and analyzed for concentration of bortezomib. FIG. 9 shows the concentration, in μg/mL, of the drug upon administration of liposomes comprised of 20:0PC/mPEG-DSPE (95/5, formulation no. 4, squares), C22:0PC/mPEG-DSPE (95/5, formulation no. 6, triangles), C24:0PC/mPEG-DSPE (95/5, formulation nos. 7 and 8, open and closed circles, respectively). A control group of animals received in intravenous injection of bortezomib in free form (diamonds). The blood circulation lifetime of bortezomib was significantly increased, relative to the free drug blood circulation lifetime, when the drug was entrapped in liposomes having a bilayer comprised of a phosphocholine phospholipid. In particular, liposomes that included C22:0PC as a primary bilayer component provided a long blood circulation time, slightly better than that provided by the liposomes with a C24:0PC lipid.

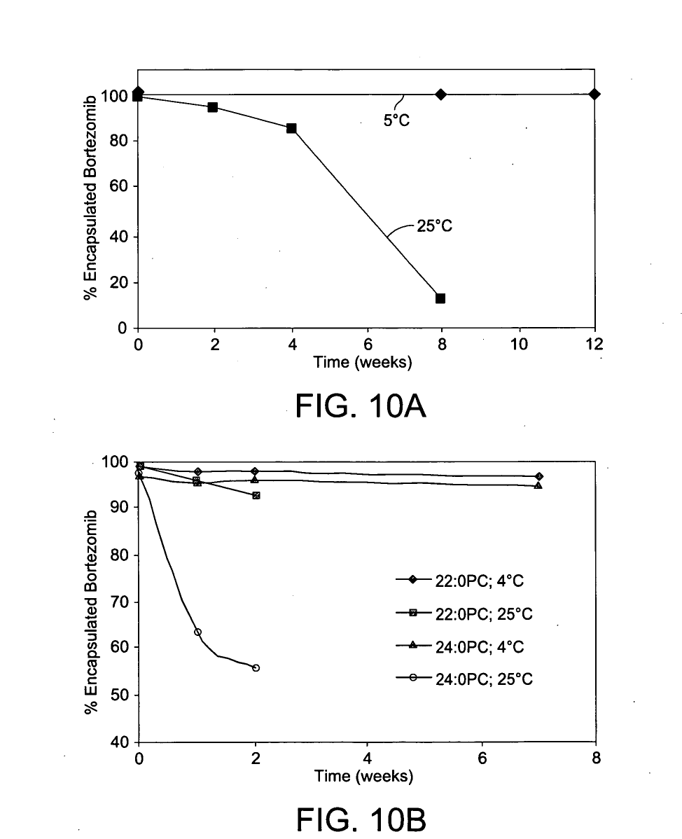

FIGS. 10A-10B show the retention of bortezomib entrapped in liposomes composed of C22:0PC/mPEG-DSPE (95/5) or of C24:0PC/mPEG-DSPE (95/5). More specifically, FIG. 10A shows the percent bortezomib encapsulation in liposomes composed of C22:0PC/mPEG-DSPE (95/5) as a function of time, in weeks, when stored at 5° C. (diamonds) or at 25° C. (squares). At 5° C., the formulation was stable for at least three months, with essentially no measurable amount of drug loss. When stored at 25° C., the drug began leaking from the liposomes after about 2 weeks of storage.

FIG. 10B shows the percent bortezomib encapsulation in liposomes composed of C22:0PC/mPEG-DSPE (95/5, diamonds, squares) or of C24:0PC/mPEG-DSPE (95/5, triangles, circles) as a function of time, in weeks, when stored at 4° C. (diamonds, triangles) or at 25° C. (squares, circles). Liposomes composed of phosphocholine with a C22:0 chain length offered better drug retention at both temperatures than liposomes composed of phosphocholine with a C24:0 chain length.

Accordingly, in one embodiment, liposomes comprised of a phospholipid having 20, 21, or 22 carbon atoms is contemplated. The lipid can be an asymmetric lipid, wherein the two acyl chains have a different carbon chain length or a symmetric lipid, where the two acyl chains have the same number of carbon atoms. In embodiments where the lipid is asymmetric, the phospholipid is considered to have 20, 21, or 22 carbon atoms when one of the two acyl chains has 20, 21, or 22 carbon atoms. In a preferred embodiment, the opposing chain has a number of carbon atoms that differs by less than 4, more preferably less than 2 carbon atoms.

Phospholipids are known in the art to be vesicle-forming lipids, as they spontaneously form into bilayer vesicles in water, with the hydrophobic moiety (acyl chain) in contact with the interior, hydrophobic region of the bilayer membrane, and the head group moiety oriented toward the exterior, polar surface of the bilayer. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, where the two hydrocarbon chains are typically between about 14-22 carbon atoms in length, and have varying degrees of unsaturation.

Vesicle-forming lipid undergo a transition from a liquid crystalline phase to a more fluid phase at a certain phase transition, or Tm, that depends on the structure of the lipid. In one embodiment, liposomes are formed from a lipid having a certain Tm, and drug is loaded into the liposomes against an ion gradient by incubating the liposomes in the presence of drug at a temperature that is below the Tₘ of that lipid, which is typically the primary lipid component in the lipid bilayer. This method of preparation is set forth generally below, and is illustrated by the liposome formulations prepared as described in Examples 3-6, where remote loading of bortezomib into liposomes was achieved at room temperature.

The remote loading of the boronic acid compound, in one embodiment, is conducted using pre-formed liposomes containing meglumine. Meglumine is a secondary amine compound, and forms a boronate ester with its diol functionalities with the boronic acid compound. The multiple vicinal cis diols in meglumine react with the boronic acid compound after it diffuses across the liposome lipid bilayer membrane, to form a boronate ester, thus entrapping the boronic acid compound in the liposome.

In one embodiment, the process is driven by pH, where a lower pH (e.g. pH 6-7) outside the liposome and somewhat higher pH (pH 8.5-10.5) on the interior of the liposome, combined with the presence of a polyol, induces accumulation and loading of the compound. In this embodiment, the composition is prepared by formulating liposomes having a higher-inside/lower-outside gradient of a polyol. An aqueous solution of the polyol, selected as described above, is prepared at a desired concentration, determined as described above. It is preferred that the polyol solution has a viscosity suitable for lipid hydration. The pH of the aqueous polyol solution is preferably greater than about 8.0 when a buffering reagent is employed to generate the internal high pH. The pH of the hydration solution containing acetic acid (or other membrane permeable weak acids) is usually at neutral, and in this case the high internal pH is generated during the process of dialysis or diafiltration.

The aqueous polyol solution is used for hydration of a dried lipid film, prepared from the desired mixture of vesicle-forming lipids, non-vesicle-forming lipids (such as cholesterol, DOPE, etc.), lipopolymer, such as mPEG-DSPE, and any other desired lipid bilayer components. A dried lipid film is prepared by dissolving the selected lipids in a suitable solvent, typically a volatile organic solvent, and evaporating the solvent to leave a dried film. The lipid film is hydrated with a solution containing the polyol, adjusted to a desired pH to form liposomes.

After liposome formation, the liposomes can be sized to obtain a population of liposomes having a substantially homogeneous size range, typically between about 0.01 to 0.5 microns, more preferably between 0.03-0.40 microns and even more preferably between 0.08-0.2 microns. One effective sizing method for REVs and MLVs involves extruding an aqueous suspension of the liposomes through a series of polycarbonate membranes having a selected uniform pore size in the range of 0.8 to 0.05 micron, typically 0.8, 0.4, 0.2, 0.1, 0.08 and/or 0.05 microns. The pore size of the membrane corresponds roughly to the average sizes of liposomes produced by extrusion through that membrane, particularly where the preparation is extruded two or more times through the same membrane. Homogenization methods are also useful for down-sizing liposomes to sizes of 100 nm or less (Martin, F. J., in Specialized Drug Delivery Systems—Manufacturing and Production Technology, P. Tyle, Ed., Marcel Dekker, New York, pp. 267-316 (1990)).

After sizing, unencapsulated bulk phase polyol is removed by a suitable technique, such as dialysis, diafiltration, centrifugation, size exclusion chromatography or ion exchange to achieve a suspension of liposomes having a high concentration of polyol inside and preferably little to no polyol outside. Also after liposome formation, the external phase of the liposomes is adjusted, by titration, dialysis or the like, to a pH of less than about 7.0.

The boronic acid compound to be entrapped is then added to the liposome dispersion for active loading into the liposomes. The amount of boronic acid compound added may be determined from the total amount of drug to be encapsulated, assuming 100% encapsulation efficiency, i.e., where all of the added compound is eventually loaded into liposomes in the form of boronate ester.

The mixture of the compound and liposome dispersion are incubated preferably at a temperature lower than the phase transition temperature of the primary lipid component in the lipid mixture forming the lipid bilayer. Uptake of the compound to a compound concentration in the liposomes that is several times that of the compound in the bulk medium is desired, and often is evidenced by the formation of precipitate in the liposomes. The latter may be confirmed, for example, by standard electron microscopy or X-ray diffraction techniques. For high-phase transition lipids having a Tₘ of 55° C., for example, incubation may be carried out at between 20-45° C. The incubation time may vary from between a few minutes, to tens of minutes, to hours or less to up to 12 hours or more, depending on incubation temperature and the strength of the complexing reagent inside the liposome. The drug loading time also depends in part on the form of the drug that is added to the liposome for loading. For example, a shorter time is required when solubilized drug is added.

At the end of this incubation step, the suspension may be further treated to remove free (non-encapsulated) compound, e.g., using any of the methods mentioned above for removing free polymer from the initial liposome dispersion containing entrapped polyol.

The liposomes can optionally include a vesicle-forming lipid covalently linked to a hydrophilic polymer. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating. Polymer-derivatized lipids comprised of methoxy(polyethylene glycol) (mPEG) and a phosphatidylethanolamine (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1,000, 2,000, 3,000, 5,000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556; Zalipsky, S. et al., Bioconjugate Chem. 8:111 (1997); Zalipsky, S. et al., Meth. Enzymol. 387:50 (2004). These lipopolymers can be prepared as well-defined, homogeneous materials of high purity, with minimal molecular weight dispersity (Zalipsky, S. et al., Bioconjugate Chem. 8:111 (1997); Wong, J. et al., Science 275:820 (1997)). The lipopolymer can also be a “neutral” lipopolymer, such as a polymer-distearoyl conjugate, as described in U.S. Pat. No. 6,586,001, incorporated by reference herein.

When a lipid-polymer conjugate is included in the liposomes, typically between 1-20 mole percent of the lipid-polymer conjugate is incorporated into the total lipid mixture (see, for example, U.S. Pat. No. 5,013,556). The liposomes can additionally include a lipopolymer modified to include a ligand, forming a lipid-polymer-ligand conjugate, also referred to herein as a ‘lipopolymer-ligand conjugate’. The ligand can be a therapeutic molecule, such as a drug or a biological molecule having activity in vivo, a diagnostic molecule, such as a contrast agent or a biological molecule, or a targeting molecule having binding affinity for a binding partner, preferably a binding partner on the surface of a cell. A preferred ligand has binding affinity for the surface of a cell and facilitates entry of the liposome into the cytoplasm of a cell via internalization. A ligand present in liposomes that include such a lipopolymer-ligand is oriented outwardly from the liposome surface, and therefore available for interaction with its cognate receptor.

Methods for attaching ligands to lipopolymers are known, where the polymer can be functionalized for subsequent reaction with a selected ligand. (U.S. Pat. No. 6,180,134; Zalipsky, S. et al., FEBS Lett. 353:71 (1994); Zalipsky, S. et al., Bioconjugate Chem. 4:296 (1993); Zalipsky, S. et al., J. Control. Rel. 39:153 (1996); Zalipsky, S. et al., Bioconjugate Chem. 8(2):111 (1997); Zalipsky, S. et al., Meth. Enzymol. 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

The ligand can be a molecule that has binding affinity for a cell receptor or for a pathogen circulating in the blood. The ligand can also be a therapeutic or diagnostic molecule, in particular molecules that when administered in free form have a short blood circulation lifetime. In one embodiment, the ligand is a biological ligand, and preferably is one having binding affinity for a cell receptor. Exemplary biological ligands are molecules having binding affinity to receptors for CD4, folate, insulin, LDL, vitamins, transferrin, asialoglycoprotein, selecting, such as E, L, and P selecting, Flk-1,2, FGF, EGF, integrins, in particular, α₄β₁ αᵥβ₃, αᵥβ₁ αᵥβ₅, αᵥβ₆ integrins, HER2, and others. Preferred ligands include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. It will be appreciated that a cell surface receptor, or fragment thereof, can serve as the ligand. Other exemplary targeting ligands include, but are not limited to vitamin molecules (e.g., biotin, folate, cyanocobalamine), oligopeptides, oligosaccharides. Other exemplary ligands are presented in U.S. Pat. Nos. 6,214,388; 6,316,024; 6,056,973; and 6,043,094, which are herein incorporated by reference.

Liposome formulations that include a lipid-polymer-ligand targeting conjugate can be prepared by various approaches. One approach involves preparation of lipid vesicles that include an end-functionalized lipid-polymer derivative; that is, a lipid-polymer conjugate where the free polymer end is reactive or “activated” (see, e.g., U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, e.g., U.S. Pat. Nos. 6,224,903 and 5,620,689). In yet another approach, a micellar solution of the lipid-polymer-ligand conjugate is incubated with a suspension of liposomes and the lipid-polymer-ligand conjugate is inserted into the pre-formed liposomes (see, e.g., U.S. Pat. Nos. 6,056,973 and 6,316,024).

### III. Methods of Use

The liposome formulations having a peptide boronic acid compound entrapped in the form of a boronate ester are used for treatment of tumor-bearing patients. Boronic acid compounds are in the class of drugs referred to as proteasome inhibitors. Proteasome inhibitors induce apoptosis of cells by their ability to inhibit cellular proteasome activity. More specifically, in eukaryotic cells, the ubiquitin-proteasome pathway is the central pathway for protein degradation of intracellular proteins. Proteins are initially targeted for proteolysis by the attachment of a polyubiquitin chain, and then rapidly degraded to small peptides by the proteasome and the ubiquitin is released and recycled.

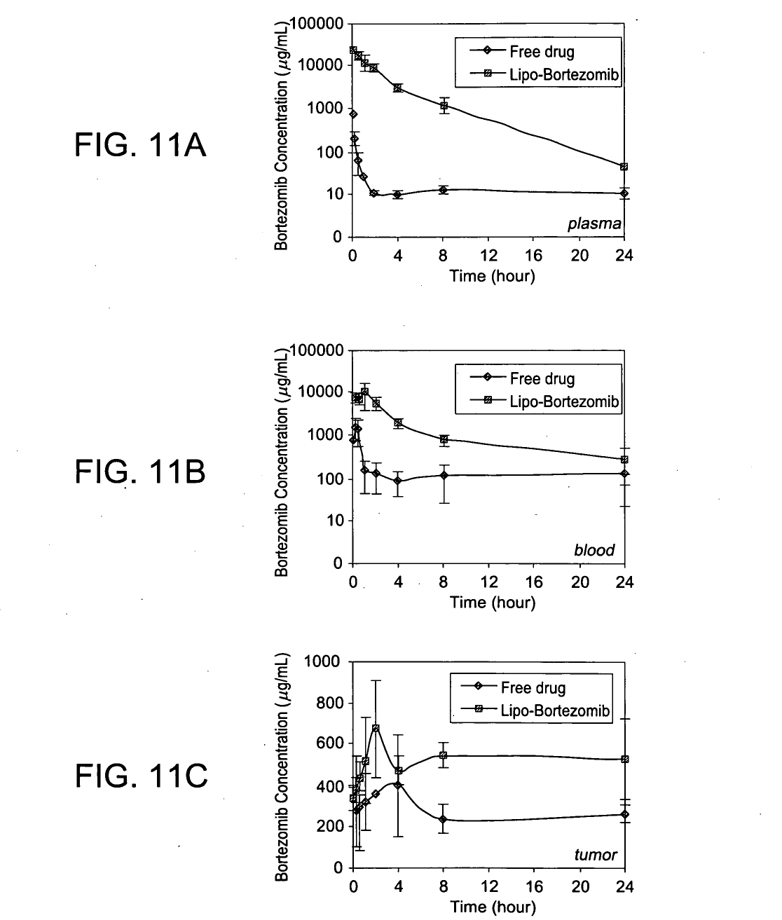

Liposome formulations prepared as described herein were administered in vivo to mice. As described in Example 7, liposomes comprised of 22:0PC/mPEG-DSPE (95/5) and containing entrapped bortezomib were prepared and administered intravenously to tumor-bearing mice. A control group of mice was treated with bortezomib sold under the trade name VELCADE®, which is a mixture of bortezomib in mannitol. FIGS. 11A-11C show the concentration, in ng/mL, of bortezomib in plasma (FIG. 11A), blood (FIG. 11B) and tumor (FIG. 11C) for the drug in free form (diamonds) or entrapped in liposomes (squares). The concentration of bortezomib in plasma, blood, and tumor was higher at all time points when administered in liposome-entrapped form than when administered as a free drug. This study shows the enhanced drug accumulation in tumor provided by the liposome formulation.

The pharmacokinetic parameters are summarized in Table 1.

TABLE 1

| | Bortezomib | Cₘₐₓ | AUC_{(0-24 h)} | | Vss-obs* | CL-obs* | CL₍ₐₚₚ₎ = dose/AUC_{24 h} |
|---|---|---|---|---|---|---|---|
| Tissue | Formulation | (ng/mL) | (hr · ng/mL) | T_{1/2}(α) | (mL/hr/kg) | (mL/hr/kg) | (mL/hr/kg) |
| Plasma | Liposome- | 730 | 485 | 0.35 | 11300 | 1630 | |
| | entrapped | | | | | | |
| | Free Drug | 23500 | 59800 | 2.7 | 42.7 | 13.3 | |
| Whole | Liposome- | 1650 | 3760 | | | | 213 |
| Blood | entrapped | | | | | | |
| | Free Drug | 10300 | 37200 | | | | 21.5 |
| Tumor | Liposome- | 462 | 6630 | | | | |
| | entrapped | | | | | | |
| | Free Drug | 674 | 12800 | | | | |

*Vss and CL were estimated using T½α phase.

The plasma area-under-the curve for liposome-entrapped bortezomib was 132 fold higher than the AUC for the free form of the drug; the plasma half life for liposome-entrapped bortezomib was 8 fold higher than the plasma half-life for the free form of the drug; the whole blood Cₘₐₓ and AUC for liposome-entrapped bortezomib were 6.2 fold and 10 fold higher, respectively, than the Cₘₐₓ and AUC for the free form of the drug; the Cₘₐₓ and AUC in the tumor for liposome-entrapped bortezomib were 1.5 fold and 1.9 fold higher, respectively, than the Cₘₐₓ and AUC for the free form of the drug.

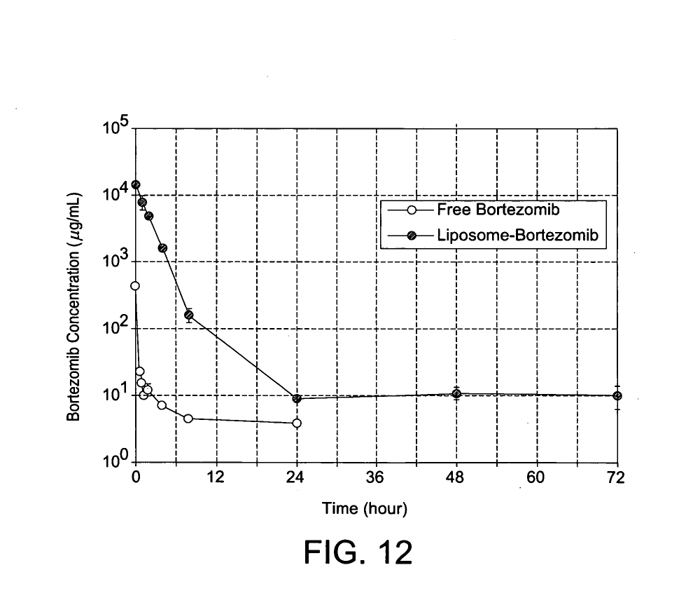

In another study, liposome-entrapped bortezomib was administered to mice and the pharmacokinetic parameters were determined. The liposomes were composed of 22:0PC/mPEG-DSPE and were prepared as described in Example 7. The plasma pharmacokinetic profiles of the liposome-entrapped bortezomib (closed circles) and of free bortezomib (open circles) are shown in FIG. 12, and the pharmacokinetic parameters are summarized in Table 2.

TABLE 2

| Bortezomib | Conc. At 5 min. | AUC_{(0-24 h)} | Vss | CL-obs* |
|---|---|---|---|---|
| Formulation | (ng/mL) | (hr · ng/mL) | (ml) | (mL/hr) |
| Free Drug | 423.3 ± 33.6 | 271 | 370 | 53.8 |
| Liposome- | 14067 ± 513 | 29138 | 1.53 | 0.55 |
| entrapped | | | | |

Accordingly, in one embodiment, a liposome formulation comprising a peptide boronic acid compound is used for treatment of cancer, and more particularly for treatment of a tumor in a cancer patient.

Multiple myeloma is an incurable malignancy that is diagnosed in approximately 15,000 people in the United States each year (Richardson, P. G. et al., Cancer Control. 10(5):361 (2003)). It is a hematologic malignancy typically characterized by the accumulation of clonal plasma cells at multiple sites in the bone marrow. The majority of patients respond to initial treatment with chemotherapy and radiation, however most eventually relapse due to the proliferation of resistant tumor cells. In one embodiment, the invention provides a method for treating multiple myeloma by administering a liposome formulation comprising a peptide boronic acid compound entrapped in the form a boronate ester.

The liposome formulation is also effective in breast cancer treatment by helping to overcome some of the major pathways by which cancer cells resist the action of chemotherapy. For example, signaling through NF-kB, a regulator of apoptosis, and the p44/42 mitogen-activated protein kinase pathway, can be anti-apoptotic. Since proteasome inhibitors block these pathways, the compounds are able to activate apoptosis. Thus, the invention provides a method for treating a subject having breast cancer, by administering liposomes comprising a peptide boronic acid compound. Moreover, since chemotherapeutic agents such as taxanes and anthracyclines have been shown to activate one or both of these pathways, use of a proteasome inhibitor in combination with conventional chemotherapeutic agents acts to enhance the antitumor activity of drugs, such as paclitaxel and doxorubicin. Thus, in another embodiment, the invention provides a treatment method where a chemotherapeutic agent, in free form or in liposome-entrapped form, is administered in combination with a liposome-entrapped peptide boronic acid compound.

Doses and a dosing regimen for the liposome formulation will depend on the cancer being treated, the stage of the cancer, the size and health of the patient, and other factors readily apparent to an attending medical caregiver. Moreover, clinical studies with the proteosome inhibitor bortezomib, Pyz-Phe-boroLeu (PS-341), provide ample guidance for suitable dosages and dosing regimens. For example, given intravenously once or twice weekly, the maximum tolerated dose in patients with solid tumors was 1.3 mg/m² (Orlowski, R. Z. et al., Breast Cancer Res. 5:1-7 (2003)). In another study, bortezomib given as an intravenous bolus on days 1, 4, 8, and 11 of a 3-week cycle suggested a maximum tolerated dose of 1.56 mg/m² (Vorhees, P. M. et al., Clinical Cancer Res. 9:6316 (2003)).

The liposome formulation is typically administered parenterally, with intravenous administration preferred with subcutaneous administration as a preferred alternative. It will be appreciated that the formulation can include any necessary or desirable pharmaceutical excipients to facilitate delivery.

In the treatment methods described above, a preferred proteosome inhibitor is bortezomib, Pyz-Phe-boroLeu; Pyz: 2,5-pyrazinecarboxylic acid; PS-341), having the structure:

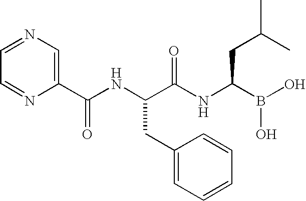

Bortezomib has been shown to have activity against a variety of cancer tissues, including breast, ovarian, prostate, lung, and against various tumors, such as pancreatic tumors, lymphomas and melanoma. (Teicher, B. A. et al., Clin Cancer Res., 5(9):2638-45 (1999); Adams, J., Semin. Oncol., 28(6):613-19 (2001); Orlowski, R. Z.; Dees, E. C., Breast Cancer Res 5(1):1-7 (2002); Frankel et al., Clin. Cancer Res. 6(9):3719-28 (2000); and Shah, S. A. et al., J Cell Biochem, 82(1):110-22 (2001)).

### IV. Examples

The following examples further illustrate the invention described herein and are in no way intended to be limiting.

### Example 1

### Loading of Bortezomib into Liposomes Using Sorbitol as Complexing Reagent

A mixture of hydrogenated soy phosphatidylcholine (HSPC), cholesterol, and polyethylene glycol-distearoylphosphatidylethanolamine (PEG-DSPE, PEG molecular weight 2,000 Da, Avanti Polar Lipids, Birmingham, Ala.) in a molar ratio of 50:45:5 was dissolved in ethanol. The lipid was then hydrated with hydration buffer of 400 mM sorbitol and 100 mM Tris buffer, pH 8.5. The final hydrated lipid suspension contained 10% (w/v) ethanol. The lipid dispersion was extruded under pressure through two, stacked Nucleopore (Pleasanton, Calif.) membranes with pore size 0.2 μm.

The outer buffer was exchanged by dialysis for a buffer of 150 mM NaCl/100 mM sodium hydroxyethylpiperazine-ethane sulfonate (HEPES) at pH 7.0.

Powdered bortezomib was added to the liposome suspension to a concentration of 3.4 mg/mL and the mixture was incubated at 65° C. with shaking for various times, ranging from 10 minutes to 7 hours.

After the incubation time, the liposomes were inspected to determine extent of entrapped bortezomib by gel chromatography on Sepharose CL-4B (Pharmacia, Piscataway, N.J.). No detectable amount of drug was entrapped in the liposomes.

### Example 2

### Loading of Bortezomib into Liposomes using Gluceptate as Complexing Reagent

Liposomes were prepared as described in Example 1, except the hydration buffer was comprised of 300 mM gluceptate and 200 mM Tris, pH 8.5. Bortezomib was added to the liposome suspension at a ratio of 2.5 mg/mL bortezomib/20 mM lipid, and the mixture was incubated at 65° C. with shaking for various times, ranging from 30 minutes to 2 hours.

After the incubation time, the liposomes were assayed to determine extent of entrapped bortezomib. About 0.15 mg/mL drug was loaded into the liposomes, an encapsulation efficiency of about 7%.

### Example 3

### Loading of Bortezomib into Liposomes using Meglumine as Complexing Reagent

Liposomes were prepared as described in Example 1, except the hydration buffer was comprised of 300 mM meglumine and 100 mM Tris, pH 8.5.

Bortezomib was added to the liposome suspension at a ratio of 2.5 mg/mL bortezomib/20 mM lipid, and the mixture was incubated with shaking, for various times of 30 minutes, 60 minutes, and 120 minutes (at 65° C.) or for 3 days at room temperature.

After incubation, the liposomes were inspected to determine extent of entrapped bortezomib. Results are shown in FIGS. 4A-4B. No drug loading was detected when the incubation was conducted at 65° C. (FIG. 4A). Liposomes incubated at room temperature with drug had about 0.3 mg/mL entrapped drug, an encapsulation efficiency of about 16% (FIG. 4B).

### Example 4

### Loading of Bortezomib into Liposomes Containing Meglumine and Acetic Acid

Liposomes were prepared as described in Example 1, except the hydration buffer was comprised of 300 mM meglumine and 300 mM acetic acid pH 7. Powdered bortezomib was added to the liposome suspension at final concentrations of 1.88 mg/mL bortezomib in approximately 100 mM lipid (lipid concentration at extrusion and not determined prior to drug loading), and the mixture was incubated at room temperature (22-25° C.), with gentle shaking, for overnight (approx. 16 hours).

After incubation, the liposomes were inspected to determine extent of entrapped bortezomib. Results are shown in FIG. 5, where an encapsulation efficiency of about 95% was achieved.

### Example 5

### Pharmacokinetic Characterization of Liposomes Containing Bortezomib

Three liposome formulations were prepared as described in Example 4, except the component concentrations were adjusted to provide the drug/lipid molar ratios set forth in the table below.

| | Loading | Drug/ | | |
|---|---|---|---|---|
| | Battery/ | Lipid | Drug | |
| Formulation | Hydration | Molar | Concentration | Encapsulation |
| No. | Buffer | Ratio | (mg/mL) | Efficiency (%) |
| 1 | meglumine/ | 65 | 0.525 | 98% |
| | acetic acid | | | |
| 2 | meglumine/ | 33 | 1.041 | 98% |
| | acetic acid | | | |
| 3 | meglumine/ | 16 | 2.132 | 99% |
| | acetic acid | | | |

Three groups of mice (n=9) were treated by intravenous injection with liposome formulation no. 1, 2, or 3. Blood samples from three mice in each group at 5 minutes, 3 hours, and 24 hours after injection. The blood was analyzed for concentration of bortezomib. Results are shown in FIG. 6.

### Example 6

### Characterization of Liposomes Having Various Lipid Compositions

A. Egg Sphingomyelin Liposome Formulations

Liposomes were prepared as described in Example 1, except lipid mixtures of egg sphingomyelin and cholesterol (55:45), egg sphingomyelin/cholesterol/mPEG-DSPE (50:45:5) or egg sphingomyelin only were hydrated with a hydration buffer of 300 mM meglumine and 300 mM acetic acid, pH 7.0. The lipid concentration post hydration was about 100 mM.

The outer buffer of each liposome suspension was exchanged for a dialysis buffer of 150 mM NaCl/100 mM HEPES at pH 7.0.

Powdered bortezomib was added to each liposome suspension at a bortezomib concentration of 1 mg/mL. The drug loading was carried out by incubation at 20-25° C., with shaking, overnight (10-12 hours).

After incubation, the liposomes were inspected to determine extent of entrapped bortezomib. Encapsulation efficiency of at least 99% was achieved for all three formulations. The liposome particle size, determined by dynamic light scattering at 900, was 179 nm (egg sphingomyelin/cholesterol/mPEG-DSPE), 266 nm (egg sphingomyelin/cholesterol) and 139 nm (egg sphingomyelin). The drug concentration of each formulation was about 0.9 mg/mL.

The liposome compositions were added to whole rat blood in a 5/95 v/v liposome suspension/blood ratio. The drug concentration in the blood was 5.5 μg/mL. The blood/liposome mixtures were incubated at 37° C. and samples were taken at 1 hour, 3 hours, 6 hours, and 24 hours, centrifuged at 5,000 rpm, and the supernatant was analyzed for bortezomib concentration using LC-MS. Results are shown in FIG. 7. The results indicated that the encapsulated bortezomib leaked out liposomes readily when incubated with whole blood.

B. Phosphatidylcholine Liposome Formulations

Liposomes were prepared using phosphatidylcholine lipids having 20, 22, or 24 carbon atoms in each acyl chain. The table below provides some details on the lipids, and includes the C18 (HSPC) lipid for comparison.

| | | Molecular | Phase |
|---|---|---|---|
| Lipid | | Weight | Transition |
| Abbreviation | Lipid Name | (Daltons) | (Tm, ° C.) |
| 18:0PC | 1,2-distearoyl-sn-glycero-3- | 790.1 | 55 |
| (HSPC) | phosphocholine | | |
| 20:0PC | 1,2-diarachidoyl-sn-glycero-3- | 846.27 | 66 |
| | phosphocholine | | |
| 22:0PC | 1,2-dibehenoyl-sn-glycero-3- | 902.37 | 75 |
| | phosphocholine | | |
| 24:0PC | 1,2-dilignoceroyl-sn-glycero-3- | 958.48 | 80 |
| | phosphocholine | | |

The liposome formulations having the following lipid compositions were prepared.

| | | Loading | Loading | Formulation | |
|---|---|---|---|---|---|
| Formulation | Lipid | Battery/Hydration | Particle Size | Potency | Encapsulation |
| No. | Composition | Buffer | (nm) | (mg/mL) | Efficiency (%) |
| 4 | 20:0PC/mPEG- | 300 mM meglumine/ | 141 | 0.42 | 94% |
| | DSPE (95/5) | 300 mM acetic acid | | | |
| 5 | 22:0PC/mPEG- | 300 mM meglumine/ | 228 | 0.478 | 81% |
| | DSPE (95/5) | 300 mM acetic acid | | | |
| 6 | 22:0PC/mPEG- | 400 mM meglumine/ | 104 | 0.48 | 99% |
| | DSPE (95/5) | 400 mM acetic acid | | | |
| 7 | 24:0PC/mPEG- | 400 mM meglumine/ | 116 | 0.50 | 96.5% |
| | DSPE (95/5) | 400 mM acetic acid | | | |
| 8 | 24:0PC/mPEG- | 600 mM meglumine/ | 106 | 0.50 | 66% |
| | DSPE (95/5) | 600 mM acetic acid | | | |

Powdered bortezomib was added into formulation no. 4 and no 5 and solubilized bortezomib solution in 100 mM HEPES and 50 mM NaCl, pH 6.5 was used for loading for formulation nos. 6-8. The mixture was incubated at 20-25° C., with shaking, for three days (formulation no. 4), for three days at 20-25° C. plus one hour a 50° C. (formulation no. 5), for 30 minutes at 45° C. (formulation no. 6), for 30 minutes hours at 50° C. (formulation nos. 7 and 8).

16.5 μL of liposome formulation no. 2 (Example 5) and 20 μL of liposome formulation no. 4 were each added to 950 μL whole rat blood, along with 30 μL or 33.5 μL, respectively, of buffer (100 mM HEPES, 150 mM NaCl, pH 7). As a control, 3.5 mg/mL free bortezomib was added to 950 μL whole rat blood, along with 45 μL of the buffer. The samples were incubated at 17° C. or at 37° C. and samples were taken at various times over 24 hours, centrifuged at 5,000 rpm, and the supernatant was analyzed for bortezomib concentration using LC-MS. Results are shown in FIGS. 8A-8B.

Formulation nos. 4, 6, 7, and 8 were administered intravenously to mice. Blood samples were taken at 5 minutes, 30 minutes, 1 hours, 2 hours, and 4 hours after injection. The blood plasma was analyzed for concentration of bortezomib. Results are shown in FIG. 9.

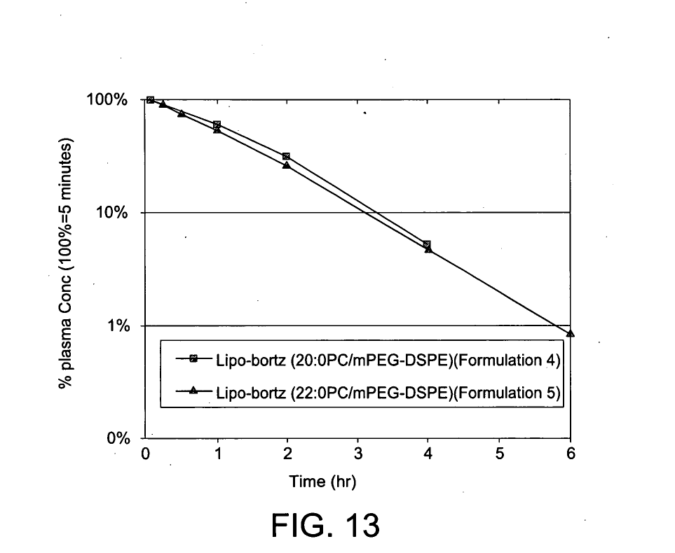

In a separate study, the pharmacokinetics of Formulation nos. 4 and 5 were evaluated in normal rats (iv bolus at 0.1 mg/kg, n=3/group). The plasma drug concentration was determined with a LC-MS assay and the results are presented in FIG. 13. The first time point was collected within 5 minutes post formulation injection. The results indicate that the liposome formulations prepared with both 20:0 PC and 22:0 PC have similar PK profiles. This result is significant because liposome formulations prepared using 20 carbon acyl chains are preferable to those prepared using 22 carbon acyl chains in view of their reduced drug and lipid degradation, yet the use of the 20 carbon acyl chain lipids in the liposome formulations does not adversely affect the PK profile. Thus, there was a lower processing temperature, and liposome formulations prepared using a lower number of carbons in the acyl chains are easier to scale up.

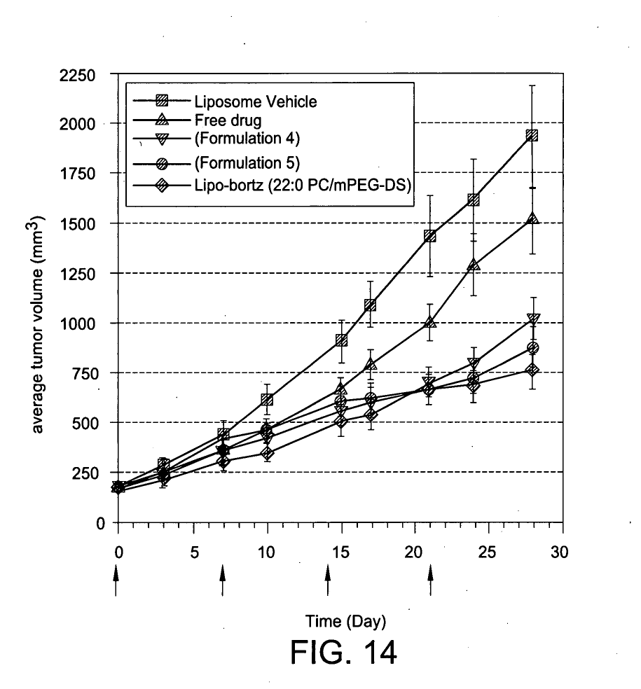

In another study, the anti-tumor efficacy of Formulations 4 and 5, and a liposomal bortazomib similar to Formulation 4, but having DS attached to PEG instead of DSPE, was evaluated in SCID mice bearing xenograft CWR22 tumors. The drug dose was 0.6 mg/kg (n=10) and was administrated intravenously weekly for four doses. The tumor size was measured and the results are shown in FIG. 14. The efficacy of all three liposomal formulations (Formulation No. 4, inverted triangles; Formulation No. 5, circles; formulation with 22:0 PC/mPEG-DS, diamonds) was significantly better than the free bortezomib (VELCADE, triangles). There was no statistical difference between the three liposomal formulations.

### Example 7

### In Vivo Activity of Liposome-Entrapped Bortezomib

A mixture of C22:0 PC and mPEG-DSPE (95/5 molar ratio) was dissolved in ethanol. The lipid solution was hydrated at 80-85° C. for 30 minutes with shaking with a hydration buffer of 400 mM meglumine, 400 mM acetic acid, at neutral to form liposomes. The lipid dispersion was extruded under pressure through two stacked Nucleopore (Pleasanton, Calif.) membranes with step-down pore sizes down to 0.1 μm.

The outer buffer of the liposome suspension was exchanged by dialysis for a buffer of 150 mM NaCl/100 mM sodium hydroxyethylpiperazine-ethane sulfonate (HEPES) at pH 7.0.

A solution of bortezomib in 100 mM HEPES and 50 mM NaCl, pH 6.5, was added to the liposome suspension at a ratio of 0.61 mg/mL bortezomib/50 mM lipid, and the mixture was incubated at 45° C., with shaking, for 30 minutes. The encapsulation efficiency was determined to be about 95%. The final drug potency post sterile filtration was 0.498 mg/mL and the lipid concentration as assayed by phosphorus assay was 52 mM. The liposome particle size post drug loading, determined by dynamic light scattering at 900, was 117 nm.

Male SCID mice bearing CWR22 tumors were randomly grouped into two test groups for treatment with intravenously administered bortezomib or liposome-entrapped bortezomib at a dose of 0.8 mg/kg. Blood and tumor samples were taken at various time points. The bortezomib concentrations in blood, plasma and tumor tissues were determined by LC-MS. Results are shown in FIGS. 11A-11C.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A liposome formulation, comprising:
liposomes comprised of a phospholipid having two acyl chains with between 20-22 carbon atoms in each chain;
a boronic acid compound entrapped in the liposomes, said compound in the form of a complex with meglumine.

2. The formulation of claim 1, wherein said phospholipid is an asymmetric phospholipid.

3. The formulation of claim 1, wherein said phospholipid is a symmetric phospholipid.

4. The formulation of claim 3, wherein said phospholipid has 20 carbon atoms.

5. The formulation of claim 1, wherein said phospholipid is a saturated phospholipid.

6. The formulation of claim 1, wherein said phospholipid is selected from the group consisting of phosphatidylcholine, phosphatidyethanolamine, phosphatidic acid, and phosphatidylinositol.

7. The formulation of claim 1, wherein said phospholipid is 1,2-arachidoyl-sn-glycero-3-phosphocholine (DAPC).

8. The formulation of claim 1, wherein said phospholipid is 1,2-dibehenoyl-sn-glycero-3-phosphocholine (DBPC).

9. The formulation of claim 1, wherein said liposomes further include a phospholipid covalently attached to a hydrophilic polymer.

10. The formulation of claim 9, wherein said hydrophilic polymer is polyethylene glycol.

11. The formulation of claim 9, wherein said phospholipid covalently attached to a hydrophilic polymer is distearoylphosphatidylethanolamine-polyethylene glycol.

12. The formulation of claim 1, wherein said boronic acid compound is a peptide boronic acid compound.

13. The formulation of claim 12, wherein said boronic acid compound is bortezomib.

14. The formulation of claim 1, wherein said liposomes further comprise entrapped acetic acid.

15. A method for preparing liposomes having an entrapped boronic acid compound, comprising
providing liposomes comprised of a phospholipid having two acyl chains, each having between 20-22 carbon atoms, said liposomes having meglumine entrapped therein;
incubating the liposomes in the presence of a boronic acid compound at a temperature lower than the phase transition temperature of the phospholipid;
whereby said incubating is effective to achieve uptake of the boronic acid compound into the liposomes.

16. The method of claim 15, wherein said providing comprises providing liposomes comprised of a phospholipid selected from the group consisting of phosphatidylcholine, phosphatidyethanolamine, phosphatidic acid, and phosphatidylinositol.

17. The method of claim 15, wherein said providing comprises providing liposomes comprised of a phospholipid selected from the group consisting 1,2-arachidoyl-sn-glycero-3-phosphocholine (DAPC) and 1,2-dibehenoyl-sn-glycero-3-phosphocholine (DBPC).

18. The method of claim 15, wherein said incubating comprises incubating in the presence of a peptide boronic acid compound.

19. The method of claim 18, wherein said peptide boronic acid compound is bortezomib.

20. The method of claim 15, wherein said providing comprises providing liposomes further comprising a phospholipid covalently attached to a hydrophilic polymer.

21. The method of claim 20, wherein said providing comprises providing liposomes having the hydrophilic polymer polyethylene glycol attached to a phospholipid.

22. The method of claim 15, whereby said incubating is effective to achieve uptake of greater than 90% of the boronic acid compound into the liposomes.

23. An improvement in a method of preparing a liposome composition comprised of liposomes comprised of a phospholipid having two acyl chains with between 20-22 carbon atoms in each chain and a boronic acid compound entrapped in the liposomes, the improvement comprising
loading the boronic acid compound into the liposomes by incubating liposomes and the boronic acid compound at a temperature below the phase transition temperature.

24. The improvement of claim 23, further comprising forming, prior to said incubating, liposomes that comprise meglumine entrapped therein.

25. The improvement of claim 23, wherein said phospholipid is 1,2-arachidoyl-sn-glycero-3-phosphocholine (DAPC) and said loading is at a temperature of between about 25-50° C.

26. The improvement of claim 23, wherein said phospholipid is 1,2-dibehenoyl-sn-glycero-3-phosphocholine (DBPC) and said loading is at a temperature of between about 25-50° C.
